(19) United States
(12) Patent Application Publication
(10) Pub. No.: US 2022/0344054 A1
(43) Pub. Date: Oct. 27, 2022

(54) STATISTICAL MODEL CREATION METHOD, STATE ESTIMATION METHOD, AND STATE ESTIMATION SYSTEM

(71) Applicant: SEMICONDUCTOR ENERGY LABORATORY CO., LTD., Atsugi-shi, Kanagawa-ken (JP)

(72) Inventors: Teppei OGUNI, Atsugi, Kanagawa (JP); Kengo AKIMOTO, Isehara, Kanagawa (JP); Tatsuya OKANO, Isehara, Kanagawa (JP)

(21) Appl. No.: 17/640,842

(22) PCT Filed: Sep. 15, 2020
(86) PCT No.: PCT/IB2020/058539

§ 371 Date: Mar. 7, 2022

(30) Foreign Application Priority Data
Sep. 27, 2019 (JP) 2019-177812

(51) Int. Cl. G16H 50/30; G06N 7/00

(52) U.S. Cl. CPC G16H 50/30; G06N 7/005

(57) ABSTRACT

A method for estimating the state of a target person in consideration of an individual difference is provided. The method includes a step of estimating the state of the target person from second data that includes speed of change in pupil area of the target person using a statistical model where a parameter is estimated from first data that includes a plurality of sets of data on speed of change in pupil area of a plurality of persons and data on the states of the plurality of persons, and a step of outputting an estimation result of the state of the target person. Note that the statistical model is a hierarchical Bayesian model using ordered logistic regression where a linear predictor is the sum of an intercept, the product of a partial regression coefficient and an explanatory variable, and a parameter showing an individual difference.

### TECHNICAL FIELD

One embodiment of the present invention relates to a statistical model creation method. Another embodiment of the present invention relates to a method for estimating a state of a target person. Another embodiment of the present invention relates to a state estimation system.

### BACKGROUND ART

Appropriate management of health conditions is an important issue. Health conditions are made worse due to fatigue accumulation. Fatigue can be classified into physical fatigue, mental fatigue, and nervous fatigue. It is comparatively easy to be aware of symptoms that appear due to physical fatigue accumulation. In contrast, in many cases, it is difficult to be aware of symptoms that appear due to mental fatigue or nervous fatigue accumulation. When mental fatigue or nervous fatigue can be quantified, the state of a target person can be judged objectively. In other words, when the fatigue level, stress condition, or the like of the target person can be estimated, the health condition of the target person can be managed appropriately.

Methods for estimating stress conditions by using machine learning or the like have attracted attention in recent years. Patent Document 1 discloses a stress judgment device that estimates a stress level from information on a target person. In addition, Patent Document 2 discloses a stress level evaluation device that acquires a value related to a stress factor based on a value related to the proportion between an iris diameter and a pupil diameter.

### REFERENCES

### Patent Documents

[Patent Document 1] Japanese Published Patent Application No. 2018-11720
[Patent Document 2] Japanese Published Patent Application No. 2008-259609

### SUMMARY OF THE INVENTION

### Problems to be Solved by the Invention

Machine learning is used in the stress judgment device disclosed in Patent Document 1. In addition, weighted values based on statistics are used for the stress factor evaluation device disclosed in Patent Document 2. Individual differences are not taken into consideration in these devices. Differences in judgment results or evaluation results between different target persons might be generated.

In view of the above, an object of one embodiment of the present invention is to estimate the state of a target person in consideration of an individual difference. Another object of one embodiment of the present invention is to provide a state estimation system in consideration of an individual difference.

Note that the description of these objects does not preclude the existence of other objects. Note that one embodiment of the present invention does not have to achieve all these objects. Note that other objects will be apparent from the descriptions of the specification, the drawings, the claims, and the like, and other objects can be derived from the descriptions of the specification, the drawings, the claims, and the like.

### Means for Solving the Problems

One embodiment of the present invention is a creation method of a statistical model used for estimating a state of a target person. The statistical model is a hierarchical Bayesian model using ordered logistic regression where a random variable is Bernoulli distribution, a link function is a logitlink function, and a linear predictor is a sum of an intercept, a product of a partial regression coefficient and an explanatory variable, and a parameter showing an individual difference. The creation method of the statistical model includes a step of inputting a dataset including a plurality of sets of data on speed of change in pupil area of a plurality of persons and data on states of the plurality of persons, a step of setting prior distribution of the intercept and prior distribution of the partial regression coefficient to non-informative prior distribution and setting prior distribution of the parameter showing the individual difference to hierarchical prior distribution, and a step of estimating posterior distribution of the intercept, posterior distribution of the partial regression coefficient, and posterior distribution of the parameter showing the individual difference by using a Markov chain Monte Carlo method.

Another embodiment of the present invention is a method for estimating a state of a target person that includes a first step of estimating the state of the target person from second data using a statistical model where a parameter is estimated from first data and a second step of outputting an estimation result of the state of the target person. The statistical model is a hierarchical Bayesian model using ordered logistic regression. The first data includes a plurality of sets of data on speed of change in pupil area of a plurality of persons and data on states of the plurality of persons. The second data includes speed of change in pupil area of the target person. The speed of change in the pupil area is an explanatory variable of the statistical model. Data on the state is a response variable of the statistical model.

In the ordered logistic regression in the above method, it is preferable that a random variable be Bernoulli distribution, a link function be a logitlink function, and a linear predictor be a sum of an intercept, a product of a partial regression coefficient and an explanatory variable, and a parameter showing an individual difference.

In addition, in the above method, it is preferable that prior distribution of the intercept and prior distribution of the partial regression coefficient be set to non-informative prior distribution, prior distribution of the parameter showing the individual difference be set to hierarchical prior distribution, and posterior distribution of the intercept, the partial regression coefficient, and the parameter showing the individual difference be estimated by using a Markov chain Monte Carlo method.

Another embodiment of the present invention is a state estimation system that includes an input portion, an output portion, an arithmetic portion, a main storage portion, and an auxiliary storage portion. The input portion has a function of inputting first data and second data. The arithmetic portion has a function of estimating a parameter of a statistical model by using the first data and creating the statistical model. The arithmetic portion has a function of estimating a state of a target person from the second data based on the statistical model. The output portion has a function of supplying information on an estimated state of the target person. The main storage portion or the auxiliary storage portion has a function of storing the statistical model. The first data includes a plurality of sets of data on speed of change in pupil area of a plurality of persons and data on states of the plurality of persons. The second data includes speed of change in pupil area of the target person.

### Effect of the Invention

According to one embodiment of the present invention, it is possible to estimate the state of a target person in consideration of an individual difference. According to another object of one embodiment of the present invention, it is possible to provide a state estimation system in consideration of an individual difference.

Note that the effects of embodiments of the present invention are not limited to the effects listed above. The effects listed above do not preclude the existence of other effects. Note that the other effects are effects that are not described in this section and will be described below. The effects that are not described in this section can be derived from the descriptions of the specification, the drawings, and the like and can be extracted from these descriptions by those skilled in the art. Note that one embodiment of the present invention has at least one of the effects listed above and/or the other effects. Accordingly, depending on the case, one embodiment of the present invention does not have the effects listed above in some cases.

### BRIEF DESCRIPTION OF THE DRAWINGS

FIG. 1A and FIG. 1B are diagrams showing hierarchical Bayesian models.

FIG. 2 is a flow chart showing an example of a method for estimating the state of a target person.

FIG. 3 is a flow chart showing an example of a method for estimating a parameter.

FIG. 4 is a flow chart showing an example of a method for estimating the state of a target person.

FIG. 5 is a flow chart showing an example of a method for estimating the state of a target person.

FIG. 6A and FIG. 6B are block diagrams each showing a structure example of a state estimation system.

### MODE FOR CARRYING OUT THE INVENTION

Embodiments will be described in detail with reference to the drawings. Note that the present invention is not limited to the following description, and it will be readily understood by those skilled in the art that modes and details of the present invention can be modified in various ways without departing from the spirit and scope of the present invention. Therefore, the present invention should not be construed as being limited to the description of embodiments below.

Note that in structures of the present invention described below, the same reference numerals are used in common for the same portions or portions having similar functions in different drawings, and a repeated description thereof is omitted. Moreover, similar functions are denoted by the same hatch pattern and are not denoted by specific reference numerals in some cases.

In addition, the position, size, range, or the like of each structure illustrated in drawings does not represent the actual position, size, range, or the like in some cases for easy understanding. Therefore, the disclosed invention is not necessarily limited to the position, size, range, or the like disclosed in the drawings.

Furthermore, ordinal numbers such as “first,” “second,” and “third” used in this specification are used in order to avoid confusion among components, and the terms do not limit the components numerically.

In this specification, the fatigue level of a target person, the stress condition of the target person, and the like are sometimes collectively referred to as the state of the target person. Thus, the term “state of target person” can be referred to as “fatigue level of target person” or “stress condition of target person.” In addition, the term “stress condition” can be sometimes referred to as the term “fatigue level.”

### Embodiment 1

In this embodiment, a method for estimating a state of a target person and a state estimation system are described. Note that the use of the method for estimating the state of the target person described in this embodiment enables detection of abnormality of the target person; thus, the method for estimating the state of the target person can also be referred to as a method for detecting abnormality of the target person. In addition, the use of the state estimation system described in this embodiment enables detection of abnormality of the target person; thus, the state estimation system can also be referred to as an abnormality detection system.

### <Method for Estimating State of Target Person>

First, the method for estimating the state of the target person is described.

A person feels fatigue due to influence of imbalance of autonomic nerves or hormones on a brain or a body. Stress can be one of the causes for imbalance of autonomic nerves or hormones. In other words, stress causes imbalance of autonomic nerves or hormones, which leads to fatigue. Therefore, imbalance of autonomic nerves (disorder of autonomic nerves) is related to fatigue or stress.

As the autonomic nerves, there are sympathetic nerves that become active at the time of body activity, during the daytime, and at the time of being nervous and parasympathetic nerves that become active at rest, at night, and at the time of being relaxed. When the sympathetic nerves become dominant, pupil dilation (mydriasis), heartbeat promotion, an increase in blood pressure, or the like occurs. In contrast, when the parasympathetic nerves become dominant, pupil contraction (miosis), heartbeat suppression, a decrease in blood pressure, or the like occurs.

In particular, it is known that mydriasis and miosis are subjected to double innervation of autonomic nerves. For example, delay in miosis is affected by excitation of sympathetic nerves and relaxation of parasympathetic nerves. In addition, for example, delay in mydriasis is affected by relaxation of sympathetic nerves and excitation of parasympathetic nerves. In particular, when parasympathetic nerves are in an excited state, miosis and mydriasis are suppressed, and the speed of change in a pupil diameter or pupil area is decreased. Accordingly, when imbalance of autonomic nerves occurs, delay occurs in miosis or mydriasis, that is, the speed of the change in the pupil diameter or pupil area is decreased.

As described above, imbalance of autonomic nerves is related to a fatigue level or a stress condition. Accordingly, it is possible to estimate the state of a target person (the fatigue level of the target person, the stress condition of the target person, or the like) from the speed of the change in the pupil diameter or pupil area of the target person. Note that the speed of the change in the pupil diameter or pupil area is treated as a numerical value. Therefore, in one embodiment of the present invention, a statistical model is used to estimate the state of the target person.

In the statistical model, setting of an explanatory variable and a response variable is important. The response variable is a variable related to a resulting matter. That is, the response variable in one embodiment of the present invention corresponds to the states of a plurality of examinees or the state of the target person. Note that the target person may be one of the plurality of examinees. Furthermore, the explanatory variable is a variable related to a cause of a problem. That is, the explanatory variable in one embodiment of the present invention corresponds to the speed of the change in the pupil diameter or pupil area.

The pupil area is proportional to the square of the pupil diameter. Thus, as compared to the case of the speed of change in the pupil diameter, it is easy to observe or acquire transition of the speed of change in the pupil area. In the following description, the speed of change in the pupil area is used as the explanatory variable. Note that in this specification, the speed of change in the pupil area can be referred to as the speed of change in the pupil diameter.

Note that the explanatory variable is not limited to the speed of change in pupil area, and the speed of change in the pupil area may be combined with any one or a plurality of temporal changes in heartbeat, a pulse, blood pressure, body temperature, a blink, posture, and the like. In addition, a questionnaire related to the state of the target person may be acquired and combined with these. Note that the temporal change in the blink refers to a temporal change in a blink time interval, a temporal change in time required for every blink, or the like.

Here, the speed of change in the pupil area refers to the degree of change in pupil area before and after a stimulus for changing the state of the examinee or the target person is applied. For example, the pupil area before the stimulus is applied is defined as the maximum pupil area, and the pupil area after the stimulus is applied is defined as the minimum pupil area. At this time, the speed of change in the pupil area is defined as a difference between the maximum pupil area and the minimum pupil area with respect to time required for changing the pupil area from the maximum pupil area into the minimum pupil area. That is, the speed of change in the pupil area corresponds to a slope when the pupil area is changed from the maximum pupil area into the minimum pupil area. Note that the maximum pupil area may be defined as the average value of the pupil area in a certain period before the stimulus is applied. In addition, the minimum pupil area may be defined as the average value of the pupil area in a certain period after the stimulus is applied.

For the state of the examinee concerning the response variable, self-evaluation is referred to. Self-evaluation with respect to the state of the examinee is acquired by a method such as taking a questionnaire.

A questionnaire survey concerning the state of the examinee is commonly examined by an interval scale or an ordinal scale. Note that data measured by an interval scale is a quantitative data, and data measured by an ordinal scale is qualitative data.

For example, stress condition surveys are conducted using two-grade evaluation of “feel stressed” and “do not feel stressed.” Alternatively, for example, the stress condition surveys are conducted using three-grade evaluation of “feel overly stressed,” “feel stressed,” and “do not feel stressed.” Note that multiple-grade evaluation such as evaluation of four or more grades may be conducted, without limitation to these.

Note that in the ordinal scale and the interval scale, a numerical value can be given on the assumption that differences between scale intervals are equal to each other. For example, in the case where stress condition surveys are conducted using three-grade evaluation, “feel overly stressed” can be evaluated as “2,” “feel slightly stressed” can be evaluated as “1,” and “do not feel stressed” can be evaluated as “0.” At this time, data measured by the ordinal scale can be regarded as quantitative data. Note that the stress condition may be measured using a stress index. For example, it is preferable to set a higher stress index as the examinee feels stressed more and set a lower stress index as the examinee feels stressed less.

As described above, since the state of the examinee is measured by the ordinal scale or the interval scale, data on the state of the examinee is discrete data. In addition, the data on the state of the examinee is represented by a non-negative integer. In other words, the data on the state of the examinee is count data. Moreover, the data on the state of the examinee is within a finite range.

There are individual differences in the states of the examinees. Examples of the individual differences include the proportion between the region of white part of the eye and the region of black part of the eye, the proportion between the region of black part of the eye and the pupil area, a difference in the speed of change in the pupil area with respect to stress, and the like. Note that the individual differences are elements that are independent of the states of the examinees. In other words, the individual differences are estimated as random effects. Accordingly, in order to take the individual differences into consideration in estimation of the state of the target person, it is preferable to use a statistical model that can take the random effects into consideration.

Therefore, in order to estimate the state of the target person in consideration of the individual differences, it is preferable to use a generalized linear mixed model (GLMM) among statistical models. The generalized linear mixed model is a statistical analysis model obtained by expansion of a generalized linear model (GLM). The generalized linear mixed model is a statistical model that can take the random effects as well as fixed effects into consideration.

The generalized linear mixed model is a statistical model that specifies probability distribution, a linear predictor, and a link function. Probability distribution refers to correspondence between the value of a random variable and its appearance probability. The linear predictor is a formula that is represented by a linear combination of a parameter and an explanatory variable. The link function is a function that leads to a linear predictor. Note that in many cases, the link function is automatically determined when probability distribution is determined.

As described above, data on the state of the examinee is acquired as count data in a finite range. Thus, it is preferable to use logistic regression as a statistical model used for estimation of the state of the target person. In particular, it is preferable to use ordered logistic regression as a statistical model in the case where the state of the examinee is acquired by multiple-grade evaluation such as evaluation of three or more grades. The ordered logistic regression is a suitable method in the case where a response variable is data on an ordinal scale or an interval scale for three or more classifications.

In this embodiment, Bernoulli distribution is used as probability distribution for ordered logistic regression. In addition, a logitlink function is used as the link function. Furthermore, the linear predictor is the sum of an intercept, the product of an explanatory variable and a partial regression coefficient, and a parameter showing an individual difference. Alternatively, the linear predictor is the sum of an intercept, the product of an explanatory variable and a partial regression coefficient, a parameter showing an individual difference, and a parameter showing an environmental difference. Note that the intercept, the partial regression coefficient, the parameter showing the individual difference, and the parameter showing the environmental difference are parameters of the statistical model. Moreover, the intercept and the partial regression coefficient are sometimes simply referred to as parameters.

In order to create a statistical model for estimating the state of the target person, it is necessary to estimate the parameter of the statistical model. Examples of a method for estimating the parameter of the statistical model include maximum likelihood estimation, maximum a posteriori estimation, Bayesian inference, and the like. Parameter estimation in maximum likelihood estimation or maximum a posteriori estimation is point estimation. In the case of this embodiment, the case where the number of data to be observed is not large is also assumed: therefore, it is preferable to use Bayesian inference in which the parameter of the statistical model is not subjected to point estimation but the probability distribution of the parameter of the statistical model is estimated.

A statistical model used for Bayesian inference (also referred to as a Bayesian statistical model) is a statistical model that has a structure where posterior distribution is proportional to the product of likelihood and prior distribution. In Bayesian inference, the probability distribution of a parameter to be estimated is calculated as posterior distribution.

It is sometimes difficult to analytically calculate posterior distribution in Bayesian inference. In the case where the posterior distribution in Bayesian inference cannot be analytically calculated, the posterior distribution in Bayesian inference can be numerically calculated. For example, numerical integration, a Markov Chain Monte Carlo (MCMC) method, or the like is preferably used. In addition, a Metropolis method, a Gibbs sampling method, or the like is preferably used as the algorithm of the MCMC method.

Among the parameters of the statistical model, the intercept and the partial regression coefficient are parameters that globally describe the entire data. In addition, the parameter showing the individual difference and the parameter showing the environmental difference are local parameters that describe only small parts of data. The global parameter is estimated using non-informative prior distribution. Furthermore, the local parameter is estimated by specifying hierarchical prior distribution.

The parameter of the statistical model in this embodiment includes at least the parameter showing the individual difference. Thus, hierarchical prior distribution is preferably used for the Bayesian statistical model in this embodiment. A Bayesian model using hierarchical prior distribution is referred to as a hierarchical Bayesian model. In this embodiment, it is preferable to create a statistical model using a hierarchical Bayesian model and numerically calculate posterior distribution using the MCMC method.

### <<Details of Method for Estimating State of Target Person>>

In this section, details of a method for estimating a state of a target person are described.

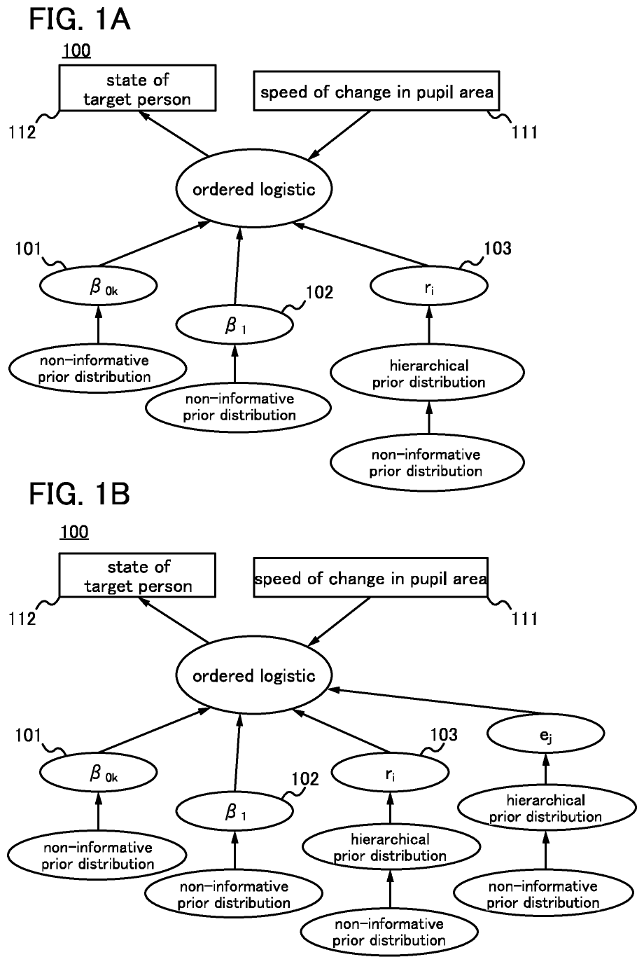

FIG. 1A is a diagram showing a hierarchical Bayesian model that is a statistical model according to one embodiment of the present invention. As illustrated in FIG. 1A, ordered logistic regression is used for a hierarchical Bayesian model 100. Note that in the ordered logistic regression, Bernoulli distribution is used as probability distribution and a logitlink function is used as a link function. In addition, a linear predictor is the sum of an intercept 101, the product of a partial regression coefficient 102 and an explanatory variable, and a parameter 103 showing an individual difference.

In addition, an explanatory variable 111 corresponds to the speed of change in the pupil area. That is, the number of explanatory variables is one. Furthermore, a response variable 112 corresponds to a stress condition that is subjected to three-grade evaluation (data classified into three). For example, “feel overly stressed” is represented as Grade 3, “feel slightly stressed” is represented as Grade 2, and “do not feel stressed” is represented as Grade 1.

First, N sets (N is a positive integer) of data on the speed of change in the pupil area of a plurality of persons and data on the stress conditions of the plurality of persons are prepared. Here, the plurality of persons are the plurality of examinees. Note that the target person may be included in the plurality of persons. In addition, the number of plurality of persons is preferably greater than or equal to two. Furthermore, the number of plurality of persons is preferably less than or equal to N. In the following description, the plurality of persons are sometimes referred to as examinees or a plurality of examinees.

Next, the stress conditions subjected to the survey of the three-grade evaluation are classified into Grade 1, and Grade 2 and Grade 3. The logistic function and the linear predictor at this time can be described as follows.

$$\begin{matrix}{q}_{i , 1} = logistic ( {η}_{i , 1} ) = \frac{1}{1 + exp ( - {η}_{i , 1} )} \\ {η}_{i , 1} = logit ( {q}_{i , 1} ) = log \frac{{q}_{i , 1}}{1 - {q}_{i , 1}} = {β}_{01} + {β}_{1} {x}_{i} + {r}_{i}\end{matrix} \qquad [ Formula 1 ]$$

Here, q_{i,1} is the probability that i-th (i is an integer of greater than or equal to 1 and less than or equal to N) data becomes Grade 2 or Grade 3. η_{i,1} is a logit of q_{i,1}. β₀₁, and β₁, and rᵢ are parameters. β₀₁ is an intercept. β₁ is a partial regression coefficient. rᵢ is a parameter showing an individual difference. xᵢ is an i-th explanatory variable, which corresponds to the speed of change in the pupil area of the i-th data. In addition, when data Y showing a stress condition here is denoted by Y₁, data showing Grade 1 is set to 0 and data showing Grade 2 or Grade 3 is set to 1 in Y₁.

Next, the stress conditions subjected to the survey of the three-grade evaluation are classified into Grade 1 and Grade 2, and Grade 3. The logistic function and the linear predictor at this time can be described as follows.

$$\begin{matrix}{q}_{i , 2} = logistic ( {η}_{i , 2} ) = \frac{1}{1 + exp ( - {η}_{i , 2} )} \\ {η}_{i , 2} = logit ( {q}_{i , 2} ) = log \frac{{q}_{i , 2}}{1 - {q}_{i , 2}} = {β}_{02} + {β}_{1} {x}_{i} + {r}_{i}\end{matrix} \qquad [ Formula 2 ]$$

In the above formula, q_{i,2} is the probability that the i-th data becomes Grade 3. η_{i,2} is a logit of q_{i,2}. β₀₂, β₁, and rᵢ are parameters. β₀₂ is an intercept. Note that the parameters other than β₀₂ are the same as β₁ and rᵢ as described above. In addition, when data Y showing a stress condition here is denoted by Y₂, data showing Grade 1 or Grade 2 is set to 0 and data showing Grade 3 is set to 1 in Y₂.

Note that in the ordered logistic regression, the sum of the probability of becoming Grade 1, the probability of becoming Grade 2, and the probability of becoming Grade 3 is 1. Thus, the probability that the i-th data becomes Grade 1 is 1-q_{i,1} and the probability that the i-th data becomes Grade 2 is q_{i,1}-q_{i,2}.

In order to create a statistical model for estimating which grade the i-th data is likely to be classified as from the input explanatory variable, it is necessary to estimate parameters β₀ₖ (k is 1 or 2), β₁, and rᵢ.

As described above, the posterior distribution of the hierarchical Bayesian model is proportional to the product of likelihood and prior distribution. In addition, the parameters β₀ₖ and β₁ are fixed effects, and the parameter rᵢ is a random effect. Thus, the following relationship is satisfied.

$$\begin{matrix}p ( {β}_{01} , {β}_{1} , s , \{ {r}_{i} \} \vert {Y}_{1} ) = \frac{p ( {Y}_{1} \vert {β}_{01} , {β}_{1} , \{ {r}_{i} \} ) p ( {β}_{01} ) p ( {β}_{1} ) p ( s ) {\prod }_{i} p ( {r}_{i} \vert s )}{p ( {Y}_{1} )} \\ p ( {β}_{02} , {β}_{1} , s , \{ {r}_{i} \} \vert {Y}_{2} ) = \frac{p ( {Y}_{2} \vert {β}_{02} , {β}_{1} , \{ {r}_{i} \} ) p ( {β}_{02} ) p ( {β}_{1} ) p ( s ) {\prod }_{i} p ( {r}_{i} \vert s )}{p ( {Y}_{2} )}\end{matrix} \qquad [ Formula 3 ]$$

Here, the left side is posterior distribution, which is probability distribution of β₀ₖ, β₁, s, and rᵢ when the data Y₁ or the data Y₂ is input. p(β₀ₖ) and p(β₁) on the right side are the intercept β₀ₖ and the prior distribution of the partial regression coefficient β₁, respectively. Here, since the intercept and the partial regression coefficient are the fixed effects, p(β₀ₖ) and p(β₁) are set to non-informative prior distribution.

Since the parameter rᵢ is the random effect, p(rᵢ|s) is set to hierarchical prior distribution. In other words, the prior distribution of rᵢ is average zero in any case and follows the normal distribution of a standard deviation s. Here, s is sometimes referred to as a super parameter. In addition, p(s) is sometimes referred to as super prior distribution. Furthermore, p(s) is set to non-informative prior distribution.

Setting is made as described above, and posterior distribution, which is the left side of the above formula, is estimated using the MCMC method. Accordingly, when the posterior distribution, which is the left side of the above formula, is estimated, posterior distribution of a plurality of parameters can be estimated.

Through the above, the parameters β₀ₖ (k is 1 or 2), β₁, s, and rᵢ can be estimated. Accordingly, the statistical model for estimating which grade the i-th data is likely to be classified as is created from the explanatory variable. For example, the speed of change in the pupil area is input as the explanatory variable and the average of the posterior distribution of β₀ₖ, β₁, and rᵢ is used so that the probability of becoming Grade 1, the probability of becoming Grade 2, and the probability of becoming Grade 3 are each calculated. Through comparison between these probabilities, it is possible to estimate which grade a fatigue level in the input speed of change in the pupil area is most likely to be classified as.

Note that as shown in FIG. 1B, a parameter eⱼ (j is a positive integer) showing an environmental difference may be added to the linear predictor. Accordingly, it is possible to estimate the stress condition in consideration of not only the individual difference but also the environmental difference.

Since the parameter eⱼ is the random effect, the prior distribution of eⱼ is average zero in any case and follows the normal distribution of a standard deviation sₚ. Furthermore, p(sₚ) is set to non-informative prior distribution.

In addition, the response variable is a stress condition subjected to (m+1)-grade (m is 3 or more) evaluation. At this time, data is classified into (m+1) pieces. In that case, data may be classified into m grades, and m sets of logistic functions and logistic models may be prepared. Note that parameters are β₀₁ to β₀ₘ, β₁, s, and rᵢ. Accordingly, when these parameters are estimated, a statistical model for estimating the stress condition can be created from the explanatory variable.

The above is the detailed description of the method for estimating the state of the target person.

Accordingly, it is possible to estimate the state of the target person in consideration of the individual difference.

### <Procedure of Method for Estimating State of Target Person>

Next, procedure of a method for estimating a state of a target person is described.

Here, the hierarchical Bayesian model is used as the statistical model for estimating the state of the target person. In addition, ordered logistic regression is preferably used for the statistical model. Furthermore, in the ordered logistic regression, Bernoulli distribution is used for the random variable and the logitlink function is used as the link function. Moreover, the linear predictor is the sum of the intercept, the product of the explanatory variable and the partial regression coefficient, and the parameter showing the individual difference. Note that the intercept and the partial regression coefficient are also parameters.

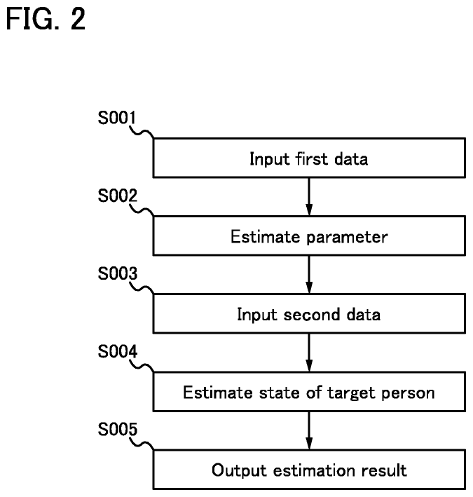

FIG. 2 is a flow chart showing an example of the method for estimating the state of the target person. The method for estimating the state of the target person includes Step S001 to Step S005 shown in FIG. 2.

Step S001 is a step of inputting first data. The first data includes a plurality of sets of data on the speed of change in the pupil area of the examinee and data on the state of the examinee (datasets). Note that the number of examinees is preferably more than one. In addition, the target person may be included in the examinee. The data on the state of the examinee include the fatigue level of the examinee, the stress condition (or stress index) of the examinee, or the like. Note that the first data may include any one or a plurality of pieces of data on temporal changes in heartbeat, a pulse, blood pressure, body temperature, a blink, posture, and the like of the examinee.

In this embodiment, the state of the target person is estimated in consideration of the individual difference. Thus, it is preferable to assign ID showing the examinee to each of the datasets included in the first data. In addition, in the case where the state of the target person is estimated in consideration of the individual difference and the environmental difference, it is preferable to assign ID showing the examinee and ID showing a measurement environment to each of the datasets included in the first data.

Step S002 is a step of estimating parameters included in the statistical model. Note that when the parameters are estimated, the statistical model for estimating the state of the target person can be created. In other words, parameter estimation can be referred to as statistical model creation.

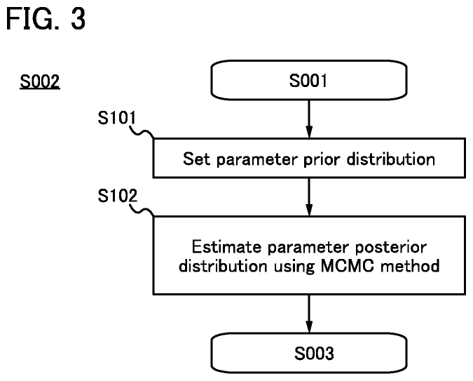

Details of Step S002 are described using FIG. 3. Step S002 includes Step S101 and Step S102.

Among the data included in the first data, the speed of change in the pupil area is used as the explanatory variable of the statistical model, and the data on the state of the examinee is used as the response variable of the statistical model. Note that in the case where the first data includes any one or a plurality of pieces of data on temporal changes in heartbeat, a pulse, blood pressure, body temperature, a blink, posture, and the like of the examinee, these pieces of data may be used as explanatory variables of the statistical model.

Step S101 is a step of setting prior distribution of the parameters. Since the intercept and the partial regression coefficient are individual effects, prior distribution of the intercept and prior distribution of the partial regression coefficient are set to non-informative prior distribution. In addition, since the parameter showing the individual difference is the random effect, prior distribution of the parameter showing the individual difference is set to hierarchical prior distribution.

Step S102 is a step of estimating posterior distribution of the parameters. For estimation of posterior distribution of the parameters, the MCMC method is preferably used.

When Step S002 (Step S101 and Step S102) is performed, the parameters included in the statistical model can be estimated. Thus, the statistical model can be created.

The above is the detailed description of Step S002.

Step S003 is a step of inputting second data. The second data needs to include the explanatory variable included in the first data. In other words, the second data includes at least the speed of change in the pupil area of the target person.

In this embodiment, the state of the target person is estimated in consideration of the individual difference. Thus, it is preferable to assign ID showing the target person to the second data. In addition, in the case where the state of the target person is estimated in consideration of the individual difference and the environmental difference, it is preferable to assign ID showing the target person and ID showing the measurement environment to the second data.

Step S004 is a step of estimating the state of the target person from the speed of change in the pupil area included in the second data. For estimation of the state of the target person, the statistical model created in Step S002 is used.

Step S005 is a step of supplying information. The information is information on the state of the target person that is estimated in Step S004. The information is supplied as, for example, visual information such as a character string, a numerical value, a graph, or a color, audio information such as voice or music, or the like.

After the information is supplied, Step S005 is terminated.

Note that in the case where the state of the target person that is estimated in Step S004 is judged normal or not abnormal, the information is not necessarily supplied. At this time, after Step S004 is terminated, the procedure may be terminated. In addition, in the case where the state of the target person is subjected to (m+1)-grade (m is 3 or more) estimation, a grade for not supplying the information may be specified in advance.

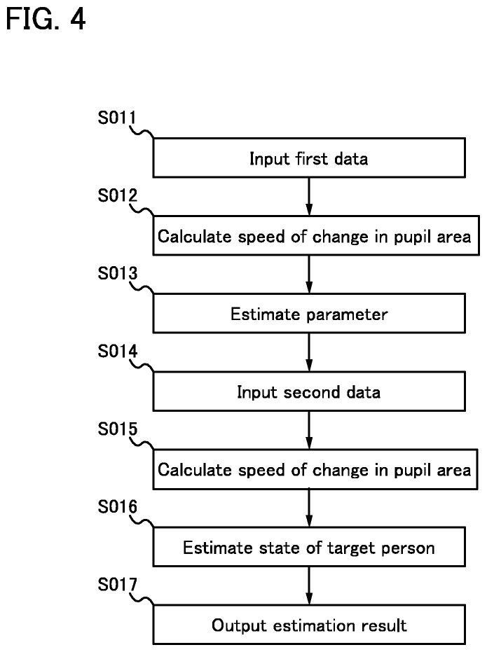

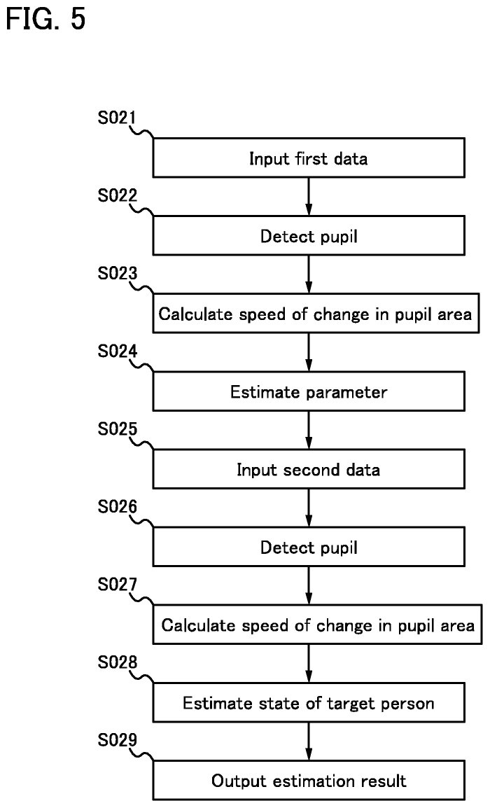

The procedure of the method for estimating the state of the target person is not limited to the above. For example, the state of the target person may be estimated according to the flow shown in FIG. 4 or FIG. 5.

FIG. 4 is a flow chart showing another example of the method for estimating the state of the target person. The method for estimating the state of the target person may include Step S011 to Step S017 shown in FIG. 4.

Step S011 is a step of inputting first data. The first data includes a plurality of sets of data on the chronological change in the pupil area of the examinee and data on the state of the examinee. Note that the number of examinees is preferably more than one. In addition, the target person may be included in the examinee. Furthermore, the first data may include any one or a plurality of pieces of data on temporal changes in heartbeat, a pulse, blood pressure, body temperature, a blink, posture, and the like of the examinee.

Step S012 is a step of calculating the speed of change in the pupil area from the chronological change in the pupil area that is included in the first data. Note that in the case where the first data includes any one or a plurality of pieces of data on temporal changes in heartbeat, a pulse, blood pressure, body temperature, a blink, posture, and the like, the speed of these changes may be calculated.

Step S013 is a step of estimating parameters included in the statistical model. Step S013 is a step similar to Step S002. Thus, Step S013 includes Step S101 and Step S102 shown in FIG. 3. The description of Step S002, Step S101, and Step S102 can be referred to for the description of Step S013.

When Step S013 is performed, the parameters included in the statistical model can be estimated. Thus, the statistical model can be created.

Step S014 is a step of inputting the second data. The second data needs to include the data included in the first data. In other words, the second data includes at least the chronological change in the pupil area of the target person. Note that in the case where the second data includes the speed of change in the pupil area of the target person, Step S015 to be described next may be omitted.

Step S015 is a step of calculating the speed of change in the pupil area from the chronological change in the pupil area that is included in the second data. Note that in the case where the second data includes any one or a plurality of pieces of data on temporal changes in heartbeat, a pulse, blood pressure, body temperature, a blink, posture, and the like of the target person, the speed of these changes may be calculated.

Step S016 is a step of estimating the state of the target person from the speed of change in the pupil area that is calculated in Step S015. For estimation of the state of the target person, the statistical model created in Step S013 is used.

Step S017 is a step of supplying information. The information is information on the state of the target person that is estimated in Step S016. The information is supplied as, for example, visual information such as a character string, a numerical value, a graph, or a color, audio information such as voice or music, or the like.

After the information is supplied, Step S017 is terminated.

Procedure of the method for estimating the state of the target person other than the above is described using FIG. 5. FIG. 5 is a flow chart showing another example of the method for estimating the state of the target person. The method for estimating the state of the target person may include Step S021 to Step S029 shown in FIG. 5.

Step S021 is a step of inputting the first data. The first data includes a plurality of sets of a moving image and data on the state of the examinee. Here, the moving image refers to a group of images for two or more frames. In addition, the moving image includes eyes of the examinee as photographic subjects. The number of examinees is preferably more than one.

Furthermore, the target person may be included in the examinee. Note that the moving image may be taken using an imaging device or may be taken by an imaging portion included in a state estimation system to be described later. Moreover, the first data may include any one or a plurality of pieces of data on temporal changes in heartbeat, a pulse, blood pressure, body temperature, a blink, posture, and the like of the examinee.

Step S022 is a step of detecting the pupil from the moving image included in the first data. In other words, Step S022 is a step of detecting the pupil from the moving image that includes the eyes of the examinee as the photographic subjects. First, a first object is detected from images included in the moving image. The first object is an eye, for example. Note that in the case where both eyes are included in the image, only one of the eyes is detected. Next, a second object is detected from the first object. The second object is a pupil, for example. Specifically, the pupil can be detected from the eye by circular extraction. Accordingly, the pupil can be detected from the moving image included in the first data.

Note that in Step S022, image processing may be performed. For example, denoising, grayscale transformation, normalization, contrast adjustment, or the like is preferably performed as image processing. Accordingly, the pupil can be detected with high accuracy.

In addition, in Step S022, machine learning is preferably performed. For example, machine learning is preferably performed using a neural network. When the pupil is detected from the moving image included in the first data using machine learning, the pupil can be detected in a short time as compared to the case where a person detects the pupil through a visual inspection, for example. In addition, even when surrounding scenery is reflected in the pupil, for example, the position of the pupil or a boundary between the pupil and an iris can be detected with high accuracy.

Step S023 is a step of calculating the speed of change in the pupil area from the pupil detected in Step S022. First, the area of the second object is calculated. Through Step S022 and the step of calculating the area of the second object, the pupil area can be calculated for each image included in the moving image. That is, the chronological change in the pupil area can be acquired.

Next, the speed of change in the pupil area is calculated from the chronological change in the pupil area. Through the above steps, the speed of change in the pupil area can be calculated from the moving image that includes the eyes of the examinee as the photographic subjects.

Note that in the case where the first data includes any one or a plurality of pieces of data on temporal changes in heartbeat, a pulse, blood pressure, body temperature, a blink, posture, and the like of the examinee, the speed of these changes may be calculated.

Step S024 is a step of estimating parameters included in the statistical model. Step S024 is a step similar to Step S002. Thus, Step S024 includes Step S101 and Step S102 shown in FIG. 3. The description of Step S002, Step S101, and Step S102 can be referred to for the description of Step S024.

When Step S024 is performed, the parameters included in the statistical model can be estimated. Thus, the statistical model can be created.

Step S025 is a step of inputting the second data. The second data needs to include the data included in the first data. In other words, the second data includes at least the moving image that includes one of the eyes of the target person as the photographic subject. Note that the moving image is preferably taken by the imaging portion included in the state estimation system to be described later. Note that in the case where the second data includes the speed of change in the pupil area of the target person, Step S026 and Step S027 to be described next may be omitted.

Step S026 is a step of detecting the pupil from the moving image included in the second data. In other words, Step S026 is a step of detecting the pupil from the moving image that includes the eyes of the target person as the photographic subjects. Note that since Step S026 is the same step as Step S022, the description of Step S022 can be referred to for the description of Step S026.

Step S027 is a step of calculating the speed of change in the pupil area from the pupil detected in Step S026. Note that since Step S027 is the same step as Step S023, the description of Step S023 can be referred to for the description of Step S027.

Note that in the case where the second data includes any one or a plurality of pieces of data on temporal changes in heartbeat, a pulse, blood pressure, body temperature, a blink, posture, and the like of the target person, the speed of these changes may be calculated.

Step S028 is a step of estimating the state of the target person from the speed of change in the pupil area calculated in Step S027. For estimation of the state of the target person, the statistical model created in Step S024 is used.

Step S029 is a step of supplying information. The information is information on the state of the target person that is estimated in Step S028. The information is supplied as, for example, visual information such as a character string, a numerical value, a graph, or a color, audio information such as voice or music, or the like.

After the information is supplied, Step S029 is terminated.

The state of the target person can be always estimated using the method for estimating the state of the target person shown in FIG. 5; thus, the health condition of the target person can be always managed. In other words, abnormality of the target person can be detected using the method for estimating the state of the target person shown in FIG. 5; thus, the health condition of the target person can be always managed.

Note that the above steps may be combined as the method for estimating the state of the target person. For example, Step S011, Step S012, Step S013, Step S025, Step S026, Step S027, Step S028, and Step S029 may be sequentially performed so that the state of the target person is estimated. Thus, since the state of the target person can be always estimated, the health condition of the target person can be always managed. In addition, the amount of data on the chronological change in the pupil area is smaller than the amount of data on the moving image; thus, the state of the target person can be estimated with high accuracy even when the amount of data is small. Accordingly, the amount of data stored in a storage portion (a main storage portion or an auxiliary storage portion) included in the state estimation system to be described later can be reduced.

The above is the description of the example of the method for estimating the state of the target person. Accordingly, it is possible to estimate the state of the target person in consideration of the individual difference.

### Structure Examples of State Estimation System

Next, structure examples of the state estimation system are described.

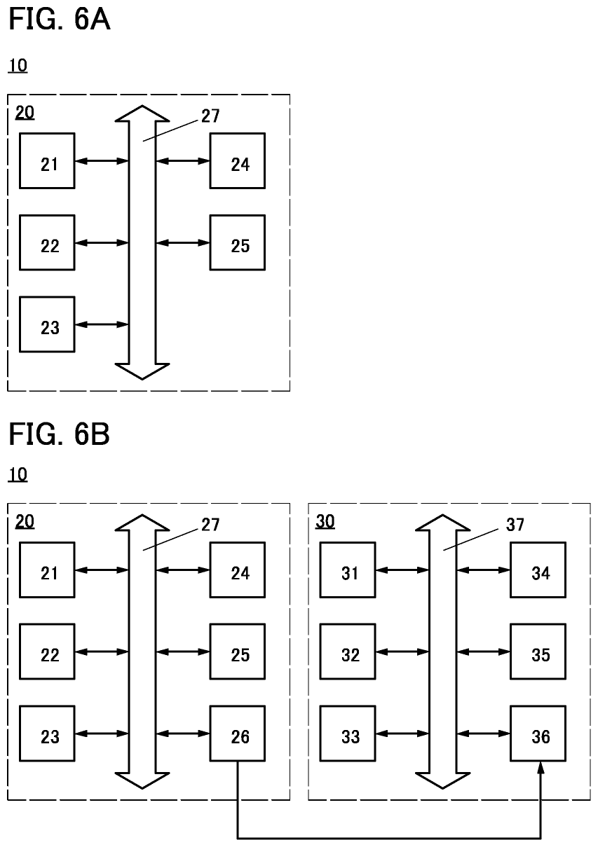

FIG. 6A is a block diagram illustrating a structure example of a state estimation system 10 that is a state estimation system according to one embodiment of the present invention. The state estimation system 10 includes an information processing device 20.

The information processing device 20 includes an input portion 21, an output portion 22, an arithmetic portion 23, a main storage portion 24, and an auxiliary storage portion 25. Data or the like can be transmitted between the components included in the information processing device 20 through a transmission path 27.

The input portion 21 has a function of inputting data. Examples of the input portion 21 include an input device such as a keyboard or a mouse. The output portion 22 has a function of supplying information.

The arithmetic portion 23 has a function of performing arithmetic processing. The arithmetic portion 23 has a function of performing predetermined arithmetic processing on data transmitted to the arithmetic portion 23 from the input portion 21, the main storage portion 24, the auxiliary storage portion 25, or the like through the transmission path 27, for example. In addition, the arithmetic portion 23 has a function of estimating a parameter and a function of estimating the state of the target person. Furthermore, the arithmetic portion 23 may have a function of processing an image included in a moving image, a function of calculating pupil area from the image, a function of calculating the speed of change in the pupil area from the chronological change in the pupil area, or the like. For example, the arithmetic portion 23 can include a CPU (Central Processing Unit), a GPU (Graphics Processing Unit), and the like.

The main storage portion 24 has a function of storing data, a program, and the like. The arithmetic portion 23 can execute arithmetic processing by reading data, a program, and the like stored in the main storage portion 24. For example, the arithmetic portion 23 can execute predetermined arithmetic processing on data read from the main storage portion 24 by executing a program read from the main storage portion 24.

The main storage portion 24 preferably operates at higher speed than the auxiliary storage portion 25. The main storage portion 24 can include a DRAM (Dynamic Random Access Memory), an SRAM (Static Random Access Memory), or the like, for example.

The auxiliary storage portion 25 has a function of storing data, a program, and the like for a longer time than the main storage portion 24. The auxiliary storage portion 25 can include an HDD (Hard Disk Drive), an SSD (Solid State Drive), or the like, for example. In addition, the auxiliary storage portion 25 may include a nonvolatile memory such as an ReRAM (Resistive Random Access Memory, also referred to as a resistance-change memory), a PRAM (Phase change Random Access Memory), an FeRAM (Ferroelectric Random Access Memory), an MRAM (Magnetoresistive Random Access Memory, also referred to a magneto-resistive memory), or a flash memory.

A statistical model that is created by parameter estimation is stored in the auxiliary storage portion 25. Note that the statistical model may be stored in the main storage portion 24.

The information processing device 20 can be provided in an information terminal such as a smartphone, a tablet, or a personal computer, for example.

Note that the information processing device 20 may include an imaging portion in addition to the above. The imaging portion has a function of performing imaging to acquire imaging data.

Note that the structure of the state estimation system 10 is not limited to the above. For example, as illustrated in FIG. 6B, the state estimation system 10 may include an information processing device 30 in addition to the information processing device 20.

The information processing device 20 includes a communication portion 26 in addition to the input portion 21, the output portion 22, the arithmetic portion 23, the main storage portion 24, and the auxiliary storage portion 25. Note that the above description can be referred to for the description of the information processing device 20 and the components included in the information processing device 20.

The communication portion 26 has a function of transmitting and receiving data or the like to and from a device or the like that is provided outside the information processing device 20. In addition, the communication portion 26 can have a function of supplying data or the like to a network and a function of acquiring the data or the like from the network.

The arithmetic portion 23 has a function of performing predetermined arithmetic processing on data transmitted to the arithmetic portion 23 from the input portion 21, the main storage portion 24, the auxiliary storage portion 25, the communication portion 26, or the like through the transmission path 27, for example.

The information processing device 30 includes an input portion 31, an output portion 32, an arithmetic portion 33, a main storage portion 34, an auxiliary storage portion 35, and a communication portion 36. Data or the like can be transmitted between the components included in the information processing device 30 through a transmission path 37.

The input portion 31 has a function of inputting data. Examples of the input portion 31 include an input device such as a keyboard or a mouse. The output portion 32 has a function of supplying information.

The arithmetic portion 33 has a function of performing arithmetic processing. The arithmetic portion 33 has a function of performing predetermined arithmetic processing on data transmitted to the arithmetic portion 33 from the input portion 31, the main storage portion 34, the auxiliary storage portion 35, the communication portion 36, or the like through the transmission path 37, for example. In addition, the arithmetic portion 33 has a function of estimating a parameter and a function of estimating the state of the target person. Furthermore, the arithmetic portion 33 may have a function of processing an image included in a moving image, a function of calculating pupil area from the image, a function of calculating the speed of change in the pupil area from the chronological change in the pupil area, or the like. For example, the arithmetic portion 33 can include a CPU, a GPU, and the like.

The main storage portion 34 has a function of storing data, a program, and the like. The arithmetic portion 33 can execute arithmetic processing by reading data, a program, and the like stored in the main storage portion 34. For example, the arithmetic portion 33 can execute predetermined arithmetic processing on data read from the main storage portion 34 by executing a program read from the main storage portion 34.

The main storage portion 34 preferably operates at higher speed than the auxiliary storage portion 35. The main storage portion 34 can include a DRAM, an SRAM, or the like, for example.

The auxiliary storage portion 35 has a function of storing data, a program, and the like for a longer time than the main storage portion 34. The auxiliary storage portion 35 can include an HDD, an SSD, or the like, for example. In addition, the auxiliary storage portion 35 may include a nonvolatile memory such as an ReRAM, a PRAM, an FeRAM, an MRAM, or a flash memory.

A statistical model that is created by parameter estimation is stored in the auxiliary storage portion 35. Note that the statistical model may be stored in the main storage portion 34.

The communication portion 36 has a function of transmitting and receiving data or the like to and from a device or the like that is provided outside the information processing device 30. For example, when data or the like is supplied from the communication portion 26 to the communication portion 36, the data or the like can be supplied from the information processing device 20 to the information processing device 30. In addition, the communication portion 36 can have a function of supplying data or the like to a network and a function of acquiring the data or the like from the network.

Here, in the case where the arithmetic portion 23 and the arithmetic portion 33 each have a function of estimating the state of the target person, the arithmetic portion 23 can create a statistical model and the created statistical model can be supplied from the information processing device 20 to the information processing device 30, for example. Accordingly, even when the arithmetic portion 33 provided in the information processing device 30 does not create a statistical model, the state of the target person can be estimated from data input to the arithmetic portion 33 on the basis of the statistical model created by the arithmetic portion 23. Consequently, the arithmetic throughput of the arithmetic portion 33 can be made lower than that of the arithmetic portion 23.

In the case where the arithmetic portion 23 creates a statistical model and the created statistical model is supplied from the information processing device 20 to the information processing device 30, the information processing device 20 can be provided in a server, for example. Note that in the case where the information processing device 20 is provided in the server, the input portion 21 and the output portion 22 are not necessarily provided in the information processing device 20. In other words, the input portion 21 and the output portion 22 may be provided outside the information processing device 20.

In addition, the information processing device 30 can be provided in an information terminal such as a smartphone, a tablet, or a personal computer, for example. Furthermore, both at least part of the components in the information processing device 20 and at least part of the components in the information processing device 30 may be provided in the server. For example, the arithmetic portion 23 and the arithmetic portion 33 may be provided in the server. In that case, for example, data acquired by the information terminal is supplied to the arithmetic portion 33 through the network, and the arithmetic portion 33 provided in the server performs estimation or the like on the data. Then, an estimation result is supplied to the information terminal through the network, so that the information terminal can acquire the estimation result.

Note that the information processing device 30 may include an imaging portion in addition to the above. The imaging portion has a function of performing imaging to acquire imaging data.

Accordingly, it is possible to provide a state estimation system in consideration of an individual difference.

Parts of the structures, methods, and the like described in this embodiment can be used in an appropriate combination.

### REFERENCE NUMERALS

10: state estimation system, 20: information processing device, 21: input portion, 22: output portion, 23: arithmetic portion, 24: main storage portion, 25: auxiliary storage portion, 26: communication portion, 27: transmission path, 30: information processing device, 31: input portion, 32: output portion, 33: arithmetic portion, 34: main storage portion, 35: auxiliary storage portion, 36: communication portion, 37: transmission path, 100: hierarchical Bayesian model, 101: intercept, 102: partial regression coefficient, 103: parameter showing individual difference, 111: explanatory variable, and 112: response variable.

1. (canceled)

2. A method for estimating a state of a target person, comprising:
a first step of estimating the state of the target person from second data using a statistical model where a parameter is estimated from first data; and
a second step of outputting an estimation result of the state of the target person,
wherein the statistical model is a hierarchical Bayesian model using ordered logistic regression,
wherein the first data includes a plurality of sets of data on speed of change in pupil area of a plurality of persons and data on states of the plurality of persons,
wherein the second data includes speed of change in pupil area of the target person,
wherein the speed of change in the pupil area is an explanatory variable of the statistical model, and
wherein data on the state is a response variable of the statistical model.

3. The method for estimating a state of a target person, according to claim 2,
wherein in the ordered logistic regression, a random variable is Bernoulli distribution,
wherein in the ordered logistic regression, a link function is a logitlink function, and
wherein in the ordered logistic regression, a linear predictor is a sum of an intercept, a product of a partial regression coefficient and an explanatory variable, and a parameter showing an individual difference.

4. The method for estimating a state of a target person, according to claim 3,
wherein prior distribution of the intercept and prior distribution of the partial regression coefficient are set to non-informative prior distribution,
wherein prior distribution of the parameter showing the individual difference is set to hierarchical prior distribution, and
wherein posterior distribution of the intercept, the partial regression coefficient, and the parameter showing the individual difference is estimated by using a Markov chain Monte Carlo method.

5. A state estimation system comprising:
an input portion;
an output portion;
an arithmetic portion; and
an storage portion,
wherein the input portion is configured to input first data and second data,
wherein the arithmetic portion is configured to create a statistical model where a parameter is estimated from the first data,
wherein the arithmetic portion is configured to estimate a state of a target person from the second data based on the statistical model,
wherein the output portion is configured to supply information on an estimated state of the target person,
wherein the storage portion is configured to store the statistical model,
wherein the first data includes a plurality of sets of data on speed of change in pupil area of a plurality of persons and data on states of the plurality of persons, and
wherein the second data includes speed of change in pupil area of the target person.
